(19) United States
(12) Patent Application Publication
(10) Pub. No.: US 2006/0004391 A1
(43) Pub. Date: Jan. 5, 2006

(54) Means for connecting blood vessels (connector of blood vessels, grafts and/or prostheses)

(76) Inventors: Axel Haverich, Isernhagen (DE); Matthias Karck, Hannover (DE); Ajay Chavan, Hannover (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 11/030,322

(22) Filed: Jan. 7, 2005

(30) Foreign Application Priority Data
Oct. 7, 2003 (DE) DE10345986.9

(51) Int. Cl. A61B 17/08

(52) U.S. Cl. 606/153

(57) ABSTRACT

The vessel connecting means or the connector for surgically connecting two vessels together comprises a tubular body having a smaller diameter in the center region than at the ends and has at least two penetrations through which pairs of circular surgical needles are inserted. The connector provides a blood-tight connection of two natural or artificial vessels, including prosthesis connections, a prosthesis with a natural vessel, or even two prostheses. The vessel connection can be made very much faster, more easily, and more reliably with the new mechanical connector than with a conventional circumferential surgical suture.

The invention concerns a means for connecting blood vessels for surgically connecting at least two blood vessels together, including both natural and artificial vessels and prostheses. Thus the invention generally concerns the field of surgical vascular connecting technologies, such as connecting two natural vessels, one vessel with one prosthesis or with a vascular connection section of that prosthesis, or connecting two prostheses together.

Two blood vessels are classically connected by a surgical suture. Various suturing techniques have been developed, such as the interrupted suture, the quilted suture, and the continuous suture. They were later transferred to connecting natural blood vessels with a vascular prosthesis. Currently, a continuous overcast [see translator's note 1] suture is generally used for the artificial connection of a blood vessel to a vascular prosthesis. A continuous suture is also used if two prostheses are connected together.

The suture material is either braided plastic or so-called monofilament strands. The latter are preferred in vascular surgery because the puncture channels which they produce are very narrow so that there is hardly any increased loss of blood on renewed circulation of blood through the vessel.

Depending on the application, suture materials that are not absorbable in the body, such as polyamides, polyethylene terephthalates, polypropylene types, and others, or materials that are absorbable in the body, such as PDS (poly-p-diaxanon), lactide copolymers, polyglactin, or others, are used.

A circumferential suture, that is, a suture around the entire circumference of the vessel, is time-consuming. On the average, the surgeon must allow between 6 and 10 minutes for it. For an arterial vascular suture, the flow of blood through the vessel must be interrupted, so that the blood supply to the tissue supplied from the artery is reduced for a time. That is not critical for most anatomical vascular surgery situations, but there are organ systems in which the tissue has only slight tolerance to reduced blood perfusion. That is particularly the case for the brain. There, and for other major artery segments, periods of clamping off that must be accepted to produce reconstructive vascular anastomoses can result in critical reduced perfusion of individual or even multiple organs.

There are also anatomic locations at which the sections of a series of vascular sutures are not all visible well or accessible with the usual surgical instrumentarium. The left Arteria subclavia is an example of a difficultly accessible region if, as is often necessary, the surgery must be accomplished through a medial sternotomy.

A classical suture can also present great difficulties if there is splicing [see translator's note 2] of the vessel wall (arterial dissection). In this case, it can happen that the arterial wall splits into two layers, neither of which can stand the tension of the needle and fiber, so that there is tearing with bleeding from the vascular anastomosis.

Thus the objective of the invention is to develop a means and a process with which two vessels (including artificial vessels and prostheses) can be connected quickly and simply, even in regions that are difficultly surgically accessible, or in case of wall weakness.

To attain this objective, the invention provides a vascular connecting means for surgically combining at least two vessels together, comprising a tubular body having at least one means for anchoring a suture for fixing the vessel or prosthesis connection to the tubular body by a ligature.

Penetrations (holes) are provided as the primary means or measure for anchoring a surgical suture to the tubular body, whereby the vessels or prosthesis connections can each be fixed with this suture, individually or jointly. The holes can be penetrated by needles or other surgical suturing tools to pull the surgical suture material through them. There can, on the other hand, also be tapered tubular segments in various embodiments which serve to anchor the suture locally in the region of tapering and prevent (lateral) slippage of the suture. The said means can also be provided multiply to the tubular body of the vascular connecting means to anchor different sutures individually, such as at a branched vascular connecting means.

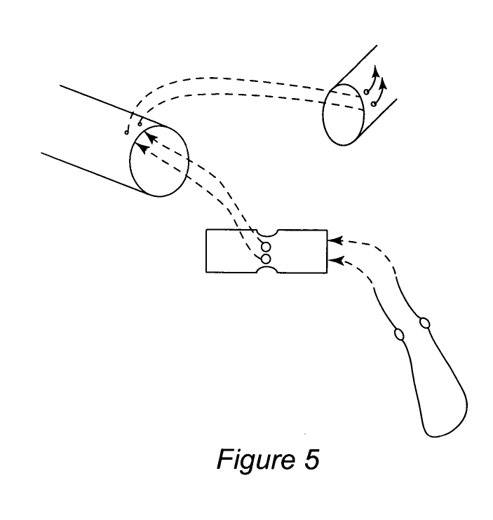

In one preferred embodiment, the means or auxiliary means for anchoring the suture comprise at least two penetrations. If there are more penetrations they are preferably multiples of two penetrations which can be stitched through in pairs by surgical needles. It is preferable for the penetrations to be in a central region of the vascular connecting means, not near the edge. They are also preferably arranged along an imaginary peripheral line around the tubular body, again preferably in groups of two. The penetrations can be utilized in pairs to produce a U suture, in which the suture material has one needle on each end (twin needles). Each one of these needles can be inserted from inside to outside, each through a penetration in the tubular body of the vessel connecting means and then through at least one vessel/one prosthesis to be connected, preferably through both of the vessels (or prostheses) to be connected. The suture fibers are then jointly knotted around the vessel and connecting means and thus the vessel to be attached or the vessels to be connected are fixed by the ligature. The ends of the suture can be knotted as usual (FIG. 5).

In another embodiment of the invention, at least two penetrations can be arranged along multiple (imaginary) circumferential lines on the tubular body. That is provided for the case in which a connection is to be made with multiple spatially separated sutures.

In addition, various tapered sections on the tubular body can be provided as means for anchoring the suture (or sutures). The means can, therefore, considered generally, consist of the external circumference of the tubular body being smaller at at least one non-terminal position than it is at both sides of at least one position that is adjacent or nearer the end.

In one preferred embodiment of the invention, having a short tubular segment with a cylindrical basic form, the external circumference at the ends of the tube is greater than it is between them, and indeed preferably initially decreases steadily from one end toward the other and then increases again. This has the advantage that a fiber wrapped around the vessel(s) prosthesis/prostheses and vessel connecting means will slip to the position of the minimum diameter or circumference. It is advantageous if an additional perforation, formed from at least two penetrations, is arranged at a position with minimum circumference in relation to its environment, because the suture can then be optimally anchored there.

It is also very advantageous if the ends of the tubular body are trumpet-shaped. The central section can have constant circumference. Also, the suture is held at its site by that, and cannot slip directly off the ends of the vessel connecting means. The trumpet-shaped ends can, at the same time, be thinner than the wall thickness of the tubular body and so can cling closely to the vessel wall. That avoids so-called “shallow-water sites [see translator's note 3]” and reduces the danger of thromboses.

According to another embodiment, the tubular body has at least one circumferential groove along an (imaginary) circumferential line, along which penetrations are preferably arranged. This groove also maintains the suture above it at its position. Several grooves can be present to be able to anchor multiple circumferential ligatures.

The tubular body is selected so that it has a somewhat smaller diameter than the vessel (or prosthesis) to be connected or the vessels (vessels and/or prostheses) to be connected at the site of the connection. It is inserted about half-way into at least one blood vessel or vascular prosthesis. Then the blood vessel or the prosthesis is punctured together with the vessel connecting means lying under it. That is preferably done with a surgical (circular) needle, with the needle inserted from the inside toward the outside through one of the penetrations (holes) which are generally roughly circular in the vessel connecting means, out from the interior of the of the sleeve or of the tubular body, and then outward through the vessel and/or the prosthesis. This stitch is done at least twice (one U-suture) or more times. The suture can be continued through further openings in the vessel connecting means if they are present. Preferably the (single or doubled) suture fibers inserted outward through the vessel or the prosthesis are wrapped circumferentially (ligated) about the prosthesis/vessel and connecting means and knotted together with a dual line of fibers so as to give a blood-tight anchoring of the vessel binding means in the prosthesis or in the vessel.

The same, or other, additional penetrations in the tube can be used to connect the second vessel or the second prosthesis with the tube, i.e, with the tubular body of the vessel connecting means. The suture is made with the usual suture material described above.

The connection between two vessels (including prostheses and prosthesis insertion sections) can preferably be done so that initially one vessel or the prosthesis is inserted at one end of the vessel connecting means and connected as described above. In a particularly preferred embodiment that is done by inserting two needles connected with suture material from the inside, first through two adjacent penetrations of the connector and then through the vessel (or prosthesis) penetrated above it. Then the first vessel (the first connection) can be fixed onto the vessel connecting means by a ligature. Alternatively both needles are further inserted through the second of the vessels to be connected so that the two overlapping perforated vessels are ligated on together.

In the case of a prosthesis-prosthesis connection, prostheses are inserted successively onto both ends of the vessel connecting means and connected as above.

Connection of a blood vessel to a prosthesis is accomplished reasonably by initially applying the prosthesis, as it can be prepared while still outside the body. That saves more expensive time. After the prosthesis has been inserted onto the vessel connecting means the prosthesis and tube are sewed through at the height of the perforations, i.e., the at least two penetrations in the tube, and then preferably tied circumferentially. Then the other end of the vessel connecting means is inserted into the blood vessel to be connected to the blood vessel and connected in the corresponding manner. The two resulting seams can overlap, especially if the same penetrations are used for both sutures, or they can lie successively (in succession in the direction of the blood flow).

A blood-tight connection can also be produced by the invention if the vessel prosthesis must be anchored at the end in a sinus. Classically (e.g., for the so-called elephant prosthesis technique) the vessel prosthesis is first inserted into the descending aorta. Then it is anchored further downstream within the aorta by a circumferential progressive suture. The ability to see into the segment of the descending aorta in which this suture must be made is very limited, so that the invention can be used here with the aortic arch open and after insertion of the vessel prosthesis of the present invention in order to produce a proximal connection of the prosthesis in the Aorta descendans immediately distal from the Aorta subclavia. Here, then, is the sequence: insertion of the vessel connecting means; sewing through at least the inner prosthesis, then the outer vessel and circumferential ligation of the vessel outside. This process can also be applied in case of aortic dissection, where the selection must be made according to the thick suture material in order to avoid the fibers cutting in during the ligation.

In one preferred embodiment, the tubular body is essentially cylindrical. Then the body has the shape of a simple cylindrical tube. However, the possibility is not ruled out that the tubular body may for certain applications conveniently be of an arc or S shape. The tubular body can also be branched, for instance, in a T or Y shape, so that it would be possible to connect three or more vessel ends together (depending on the degree of branching).

The vessel connecting means which can, for example, also be called a vascular connector, mechanical adapter or vessel connector, can be made from or consist of any mechanically suitable material allowed for use in human or animal bodies. Many such materials that are used, for example, for orthopedic and dental implants, surgical instruments, heart valves and the like, are known to those skilled in the area of surgery and implantology.

The vessel connecting means can preferably be made of at least one of the following materials: metal, especially titanium or stainless steel, including special alloys used for implants and medical instruments, carbon materials, including carbon fiber nets, soft plastic such as silicone, for example; hard plastic such as Teflon for example; ceramic material, or biologically absorbable material.

According to one possible embodiment, the tubular body of the vessel connecting means can be made of a relatively harder material and the preferably trumpet-shaped connections of a relatively soft material, with the penetrations arranged in the harder material. Then the suture is made in the region of the harder material, while the expanded ends of the vessel connector, made of the relatively soft material, allow a close fit to the vessel/prosthesis wall.

In a modification of this embodiment, the preferably trumpet-shaped connectors are connected from the relatively softer material in one piece through a central section of the relatively softer material, and a ring of the relatively harder material wrapped around the central piece or underlaid by the central piece.

The vessel connecting means according to the invention can have a coating and/or structure that partially or entirely prevents adhesion of blood components, preferably partially or entirely on the inner side, i.e., luminal.

Such a coating can consist of a material smoothing the surface or making it more slippery. For instance, a polybutylate or a mixture or copolymer with polybutylate can be used as a coating increasing the slipperiness. The coating can also contain antithrombotic medications, such as heparin. It is further possible to provide a coating that produces a lotus effect [see translator's note 4] on the surface. The lotus structure can, alternatively, also be developed directly on the surface of the tubular body, without a special coating. Coated and structured surfaces for medical devices are known, for example, from WO 00/07633 or DE 199 50 452.

The invention is described in more detail in the following, using examples shown in the drawing. The examples were selected for illustrative purposes. They should not give rise to limitation of the general possibilities of the invention. In particular, they show:

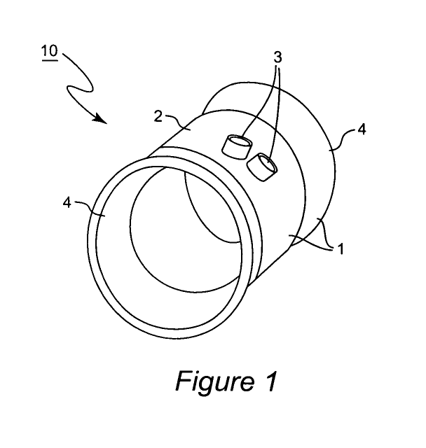

FIG. 1 a perspective view of a vessel connector with trumpet-shaped connections;

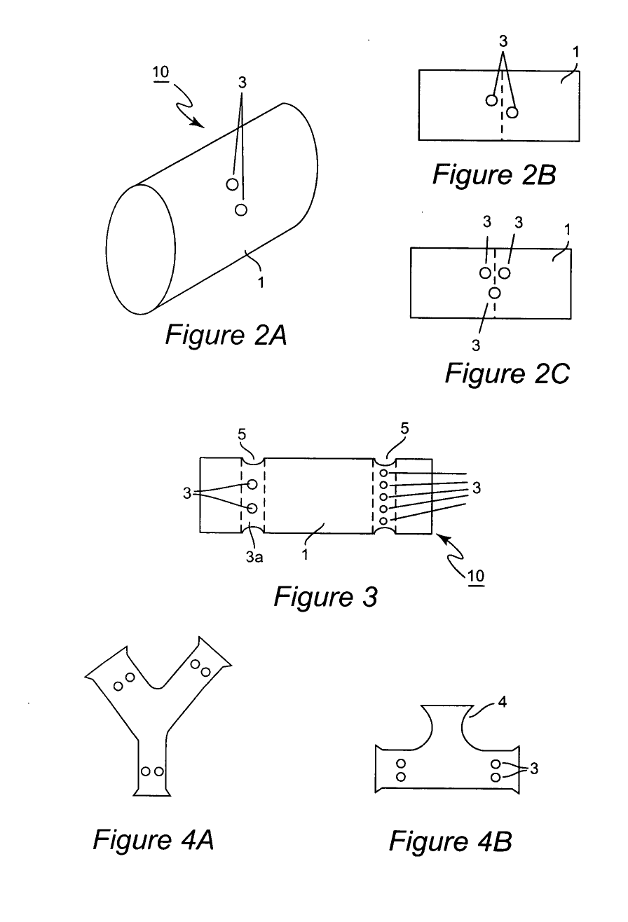

FIG. 2a a perspective view of a one-piece cylindrical vessel connector;

FIG. 2b a lateral view of a cylindrical vessel connector with staggered penetrations;

FIG. 2c a lateral view of a cylindrical vessel connector with 3 penetrations;

FIG. 3 a lateral view of a cylindrical vessel connector with two circumferential grooves;

FIG. 4a a lateral view of a Y-shaped vessel connector;

FIG. 4b a lateral view of a T-shaped vessel connector;

FIG. 5 illustrates a possible connecting procedure.

### EXAMPLES

FIG. 1 shows the vessel connecting means, designated as a whole by 10, in a perspective view. The tubular body 1 is a basically cylindrical tubular segment having a center part 2 with reduced circumference or diameter. In the present example, which shows the currently preferred embodiment, the center part 2 consists of a material that is relatively harder than the end regions, namely carbon, that is, of materials based on carbon or a cloth of carbon fibers.

The penetrations 3 are also found in a central region of this center part. In this example, there are two penetrations arranged along the central peripheral line. Multiple penetrations can also be provided, up to the extent of, for instance, a continuous perforation around the center circumference of the vessel connecting means. Furthermore, penetrations can also be arranged in two or more rows, to make it possible to use multiple adjacent sutures.

The center part 2 is surrounded here by two slightly trumpet-shaped lips 4. In the present example, the lips 4 are of a material that is relatively softer than the center part 2, namely polysulfone (PSU), which adapts or clings more easily to the vessel wall or prosthesis to be connected. The lips 4 can be glued to the center part 2 or mechanically dovetailed to it or mechanically locked to it. However, the entire tubular body 1 can also be made of a uniform material.

The lips 4 can be made conical and formed to a cylindrical center piece 2 with the diameter remaining constant over the length. Then the tubular vessel connector has a constant outside diameter in the suture region, as shown here in the drawing, and relatively expanded ends. Alternatively, the vessel connecting means can also be shaped so that the outside diameter decreases steadily from one end to the middle and increases steadily again from the middle to the other end.

The vessel connecting means 10 shown in FIG. 1 is approximately 10 mm long and has a diameter that is adapted to the diameter of the vessel or prosthesis outlet to be connected, that is, kept somewhat smaller for insertion into this vessel. The diameter of the center part 2 is correspondingly between about 4 mm and 30 mm, preferably 6 to 12 mm. The diameter of the penetrations is preferably about 1 mm or between about 0.5 mm and 1.5 mm. The figure is not drawn to scale.

FIG. 2a shows a simple cylindrical vessel connecting means 10 of a uniform material such as stainless steel or carbon. The two penetrations 3 are placed in the center part and need not lie on a circumferential line, as FIG. 2b shows. A staggered arrangement of the penetrations is also possible. The staggered arrangement, or an arrangement oblique to the circumference, can simplify the stitching. A staggered arrangement can also cause the penetration not to be under the first suture if the second suture is to be made at the same position, that is, above the first suture.

FIG. 2c shows another possible arrangement of the penetrations, here showing three penetrations.

FIG. 3 shows another embodiment of a cylindrical vessel connecting means, in which the tubular body 1, which could also be shaped as in FIG. 1 and is shown as cylindrical here only for example, also has two grooves 5. The grooves 5 indicate the desired positions for the sutures to be applied. As the two intended sutures in this example should not be superimposed and are covered with suture material by the ligatures, the penetrations 3 are placed in the grooves. That also assures that the connection does not leak blood. The vessel connecting means shown in FIG. 3 is particularly suited for connecting a prosthesis to a natural (biological) vessel: First, the prosthesis is connected, outside the body, to the left end shown in the figure. Two penetrations are sufficient for that, as described above. Exceptionally, even a single penetration 3a could suffice, as it would be adequate to stitch just once through the connector from the inside to the outside and then to wrap the suture circumferentially; that is, to wrap and tie the prosthesis lip and the vessel connecting means together. Alternatively, the procedure could be done and the connection made even without a penetration, that is, by winding a ligature around. The latter possibility is preferred only if there is, as here, a groove 5 or other narrowing to anchor the suture. Numerous penetrations 2 are provided, distributed around the circumference of the groove 5 at the right in the figure, so that the surgeon can select suitable penetrations according to the local situation.

FIG. 4a shows a Y-shaped vessel connecting means, and FIG. 4b shows a T-shaped one. The exact shape and length of the lips is variable and can be selected according to the local details of the application.

The examples with slots or narrowings and/or trumpet-shaped expansions could also be made without penetrations.

In another alternative, the entire tubular body can have a net-like texture. In particular, it can consist of a metal net or a net of medically suitable metal alloys or metal-containing compounds. Suitable net materials are known from stents, for instance. The net can have a mesh width that allows stitching through with a needle over its entirely or in certain regions. ML

1. Vessel connecting means (10) for surgically connecting at least two vessels together, having a tubular body (1) having at least one means (3;5) to anchor a suture for fixing the vessel or prosthesis connection to the tubular body by a ligature, whereby the means for anchoring the suture comprises at least two penetrations (3) through which pairs of surgical needles can be inserted, and at least two penetrations (3) arranged along one or more circumferential lines on the tubular body (1).

2. Vessel connecting means according to claim 1, characterized in that the outer circumference of the tubular body (1) is smaller at at least one non-terminal position than at both sides of at least one position nearer to or adjacent to the end.

3. Vessel connecting means according to claim 2, characterized in that the tubular body (1) extends into trumpet shapes at its ends.

4. Vessel connecting means according to claim 2, characterized in that the tubular body (1) has a groove running all the way around along at least one circumferential line on which the penetrations (3, 3a) are arranged.

5. Vessel connecting means according to claim 1, characterized in that the tubular body (1) is essentially cylindrical or is built up of essentially cylindrical tubular segments, preferably T-shaped or Y-shaped.

6. Vessel connecting means according to claim 1, characterized in that it is made of at least one of the following materials: metal, preferably titanium or stainless steel; carbon; soft plastic, especially silicone; hard plastic, especially Teflon, or biologically absorbable material.

7. Vessel connecting means (10) according to claim 1, characterized in that the tubular body (1) has an essentially cylindrical center piece (2) of a relatively harder material and preferably trumpet-shaped lips (4) of a relatively softer material, with the penetrations (3) being in the harder material.

8. Vessel connecting means according to claim 3, characterized in that the preferably trumpet-shaped lips (4) of the relatively softer material are integrally linked through a center piece of the same relatively softer material and that a ring of the relatively harder material is placed around or under the central section for the center piece (2).

9. Vessel connecting means according to claim 1, characterized in that it is provided at least partially with a coating and/or structure that prevents adhesion of blood components, preferably entirely or partially on the inner side.

10. Vessel connecting means according to claim 1, characterized in that the entire tubular body has a netlike texture, preferably of metal or metal-containing compounds.

11. Process for connecting two vessels together, in which each of two needles (twin needles) connected to surgical suture material is passed from inside to outside through one of a pair of penetrations on a vessel connecting means according to claim 1, after which both needles are stitched through an edge region/a cut edge of a first vessel, after which both needles are stitched through an edge region/cut edge of a second vessel, the two vessel ends are pulled over the vessel connector, and the suture is completed by a ligature on the vessel connector.

12. Process according to claim 11, whereby at least one of the vessels is an end of a prosthesis or a transplant that is to be connected to a vessel end.
